SEQUENCE LISTING (29 sequences)

SEQ ID NO: 1 - protein, 5 aa, synthetic construct
SYAMN

SEQ ID NO: 2 - protein, 17 aa, synthetic construct
AITMSGITAYYTDDVKG

SEQ ID NO: 3 - protein, 15 aa, synthetic construct
EEFLPGTHYYYGMDV

SEQ ID NO: 4 - protein, 11 aa, synthetic construct
RASQGIRNDLG

SEQ ID NO: 5 - protein, 7 aa, synthetic construct
SASTLQS

SEQ ID NO: 6 - protein, 9 aa, synthetic construct
LQDYNYPWT

SEQ ID NO: 7 - protein, 5 aa, synthetic construct
PYYWT

SEQ ID NO: 8 - protein, 16 aa, synthetic construct
YIHSSGYTDYNPSLKS

SEQ ID NO: 9 - protein, 11 aa, synthetic construct
ADWDRLRALDI

SEQ ID NO: 10 - protein, 11 aa, synthetic construct
RASQSIRSHLN

SEQ ID NO: 11 - protein, 7 aa, synthetic construct
GASNLQS

SEQ ID NO: 12 - protein, 9 aa, synthetic construct
QQSTGAWNW

SEQ ID NO: 13 - protein, 124 aa, synthetic construct
EVQLLESGGGLVQPGGSLRLSCAASGFTFSSYAMNWVRQAPGKGLEWVSAITMSGITAYYTDDVKGRFTISRDNSKNTLYLQMNSLRAEDTAVYYCAKEEFLPGTHYYYGMDVWGQGTTVTVSS

SEQ ID NO: 14 - protein, 107 aa, synthetic construct
AIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIK

SEQ ID NO: 15 - protein, 119 aa, synthetic construct
QVQLQESGPGLVKPSETLSLTCTVSGGSISPYYWTWIRQPPGKCLELIGYIHSSGYTDYNPSLKSRVTISGDTSKKQFSLKLSSVTAADTAVYYCARADWDRLRALDIWGQGTMVTVSS

SEQ ID NO: 16 - protein, 107 aa, synthetic construct
DIQLTQSPSSLSASVGDRVTITCRASQSIRSHLNWYQQKPGKAPKLLIYGASNLQSGVPSRFSGSGSGTDFTLTISSLQPEDFATYYCQQSTGAWNWFGCGTKVEIK

SEQ ID NO: 17 - protein, 246 aa, synthetic construct
QVQLQESGPGLVKPSETLSLTCTVSGGSISPYYWTWIRQPPGKCLELIGYIHSSGYTDYNPSLKSRVTISGDTSKKQFSLKLSSVTAADTAVYYCARADWDRLRALDIWGQGTMVTVSSGGGGSGGGGSGGGGSGGGGSDIQLTQSPSSLSASVGDRVTITCRASQSIRSHLNWYQQKPGKAPKLLIYGASNLQSGVPSRFSGSGSGTDFTLTISSLQPEDFATYYCQQSTGAWNWFGCGTKVEIK

SEQ ID NO: 18 - protein, 20 aa, synthetic construct
GGGGSGGGGSGGGGSGGGGS

SEQ ID NO: 19 - protein, 720 aa, synthetic construct [sequence omitted]

SEQ ID NO: 20 - protein, 214 aa, synthetic construct
AIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 22 - protein, 5 aa, synthetic construct
EPKSC

SEQ ID NO: 23 - protein, 10 aa, synthetic construct
DKTHTCPPCP

SEQ ID NO: 24 - protein, 107 aa, synthetic construct [sequence omitted]

SEQ ID NO: 25 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 5 aa, synthetic construct
  source 1..5: note = There may be 0, 1, 2, 3, 4 or 5 repeating units of GGGGS
  REPEAT 1..5
GGGGS

SEQ ID NO: 27 - protein, 4 aa, synthetic construct
  REPEAT 1..4: note = There may be 0, 1, 2, 3, 4 or 5 repeating units of GGGG
GGGG

SEQ ID NO: 28 - protein, 10 aa, synthetic construct
GGGGSGGGGS

SEQ ID NO: 29 - protein, 20 aa, synthetic construct
GGGGSGGGGSGGGGSGGGGS

(19) United States
(12) Patent Application Publication
(10) Pub. No.: US 2025/0382356 A1
(43) Pub. Date: Dec. 18, 2025

(54) ANTI-PCRV AND PSL BISPECIFIC ANTIBODIES FOR TREATMENT OF BRONCHIECTASIS

(71) Applicant: AstraZeneca AB, Södertälje (SE)

(72) Inventors: Antonio DIGIANDOMENICO, Gaithersburg, MD (US); Bret SELLMAN, Gaithersburg, MD (US); Wayne BRAILSFORD, Södertälje (SE); Kristoffer OSTRIDGE; Rodney Jason HUGHES, Cambridge (GB)

(21) Appl. No.: 18/878,285

(22) PCT Filed: Jun. 28, 2023
(86) PCT No.: PCT/EP2023/067575

§ 371 Date: Dec. 23, 2024

Related U.S. Application Data
(60) Provisional application No. 63/367,309, filed on Jun. 29, 2022.

(51) Int. Cl. C07K 16/12; A61K 39/00; A61P 11/00; A61P 31/04

(52) U.S. Cl. CPC C07K 16/1214; A61P 11/00; A61P 31/04; A61K 2039/505; A61K 2039/54; C07K 2317/31; C07K 2317/522; C07K 2317/524; C07K 2317/526; C07K 2317/565; C07K 2317/622

(57) ABSTRACT

This disclosure relates to anti-Pseudomonas Psl and PcrV bispecific antibodies for use in treatment of bronchiectasis.

### FIELD OF THE INVENTION

The present disclosure relates to uses of bispecific antibodies thereof that specifically bind to the Pseudomonas aeruginosa PcrV protein and Psl exopolysaccharide for treating bronchiectasis.

### BACKGROUND

Pseudomonas aeruginosa (P. aeruginosa) is a gram-negative opportunistic pathogen that causes both acute and chronic infections in compromised individuals. This is partly due to the high innate resistance of the bacterium to clinically used antibiotics, and partly due to the formation of highly antibiotic-resistant biofilms. Furthermore P. aeruginosa is known to colonize the airway in patients with non-cystic fibrosis bronchiectasis. Non-cystic fibrosis bronchiectasis is a chronic disease characterized by abnormal and permanent dilation of the bronchi resulting in chronic cough, sputum production, and recurrent bacterial infections of the airway. Patients with bronchiectasis suffer from a high morbidity due to frequent exacerbations impairing quality of life and facilitating resistance to antibiotics, leading to reduced lung function.

There is a significant unmet medical need for treatment of bronchiectasis and no approved therapy for the reduction of exacerbations is currently available. The European Respiratory Society (ERS) 2017 guidelines for the management of adult bronchiectasis suggests that apart from antibiotics to treat acute exacerbations, no other treatment can be recommended. Due to the increasing multidrug resistance which bacteria exhibit, there remains a need in the art for the development of novel strategies for the identification of new Pseudomonas-specific prophylactic and therapeutic strategies.

### BRIEF SUMMARY

Provided herein are anti-Pseudomonas Psl and PcrV bispecific antibodies for use in treatment of bronchiectasis.

In some aspects provided herein, a method of treating bronchiectasis in a subject in need thereof comprises administering to the subject a bispecific antibody that specifically binds to Pseudomonas aeruginosa PcrV protein and Psl exopolysaccharide.

In some aspects provided herein, a method of improving pre-bronchodilator forced expiratory volume 1 (FEV₁) in a subject with bronchiectasis comprises administering to the subject a bispecific antibody that specifically binds to Pseudomonas aeruginosa PcrV protein and Psl exopolysaccharide.

In some aspects provided herein, a method of reducing Pseudomonas aeruginosa load in a subject with bronchiectasis comprises administering to the subject a bispecific antibody that specifically binds to Pseudomonas aeruginosa PcrV protein and Psl exopolysaccharide.

In some aspects provided herein, a method of reducing bronchiectasis exacerbations in a subject in need thereof comprises administering to the subject a bispecific antibody that specifically binds to Pseudomonas aeruginosa PcrV protein and Psl exopolysaccharide. In some aspects, the administration reduces bronchiectasis exacerbations requiring hospitalization. In some aspects, the administration reduces bronchiectasis exacerbations requiring antibiotics. In some aspects, the administration reduces bronchiectasis exacerbations requiring hospitalization and bronchiectasis exacerbations requiring antibiotics.

In some aspects provided herein, a method of reducing the need for intravenous antibiotics in a subject with bronchiectasis comprises administering to the subject a bispecific antibody that specifically binds to Pseudomonas aeruginosa PcrV protein and Psl exopolysaccharide.

In some aspects provided herein, a method of stabilizing lung function in a subject with bronchiectasis comprises administering to the subject a bispecific antibody specifically binds to Pseudomonas aeruginosa PcrV protein and Psl exopolysaccharide.

In some aspects, the bronchiectasis is non-cystic fibrosis bronchiectasis. In some aspects, the non-cystic fibrosis bronchiectasis was confirmed by chest computed tomography (CT) demonstrating bronchiectasis affecting 1 or more lobes.

In some aspects, the subject is colonized with Pseudomonas aeruginosa. In some aspects, the respiratory tract of the subject is colonized with Pseudomonas aeruginosa. In some aspects, the Pseudomonas aeruginosa colonization has been detected by sputum culture.

In some aspects, the subject is chronically infected with Pseudomonas aeruginosa. In some aspects, the subject has airway neutrophilia. In some aspects, the subject has sputum neutrophilia. In some aspects, the subject has a history of at least 2 moderate to severe bronchiectasis exacerbations per year requiring antibiotics. In some aspects, the subject has a history of at least 1 exacerbation requiring hospital care. In some aspects, the subject is on long term nebulized antibiotics. In some aspects, the subject has chronic obstructive pulmonary disease (COPD).

In some aspects, the bronchiectasis was caused by hypogammaglobulinemia. In some aspects, the bronchiectasis was caused by common variable immunodeficiency. In some aspects, the bronchiectasis was caused by alpha-1-antitrypsin deficiency.

In some aspects, the administration reduces Pseudomonas aeruginosa in sputum cultures obtained from the subject In some aspects, the reduction occurs within 12 weeks of the first administration. In some aspects, the reduction occurs within 8 weeks of the first administration. In some aspects, the reduction occurs within 4 weeks of the first administration.

In some aspects, the administration decreases antibiotic usage. In some aspects, the administration eradicates Pseudomonas aeruginosa in the subject.

In some aspects, the bispecific competitively inhibits binding to PcrV of an antibody comprising a heavy chain variable region comprising the amino acid sequence of SEQ ID NO:13 and a light chain variable region comprising the amino acid sequence of SEQ ID NO:14. In some aspects, the bispecific antibody binds to the same epitope of PcrV as an antibody comprising a heavy chain variable region comprising the amino acid sequence of SEQ ID NO:13 and a light chain variable region comprising the amino acid sequence of SEQ ID NO:14.

In some aspects, the bispecific antibody comprises a PcrV-binding domain comprising a heavy chain CDR1 comprising the amino acid sequence of SEQ ID NO:1, a heavy chain CDR2 comprising the amino acid sequence of SEQ ID NO:2, a heavy chain CDR3 comprising the amino acid sequence of SEQ ID NO:3, a light chain CDR1 comprising the amino acid sequence of SEQ ID NO:4, a light chain CDR2 comprising the amino acid sequence of SEQ ID NO:5, and a light chain CDR3 comprising the amino acid sequence of SEQ ID NO:6. In some aspects, the bispecific antibody comprises a PcrV-binding domain comprising a heavy chain variable region comprising the amino acid sequence of SEQ ID NO:13. In some aspects, the bispecific antibody comprises a PcrV-binding domain comprising a light chain variable region comprising the amino acid sequence of SEQ ID NO:14. In some aspects, the bispecific antibody comprises a PcrV-binding domain comprising a heavy chain variable region comprising the amino acid sequence of SEQ ID NO:13 and a light chain variable region comprising the amino acid sequence of SEQ ID NO:14.

In some aspects, the bispecific antibody comprises a PcrV-binding domain with a heavy chain variable region and a light chain variable region on separate polypeptides.

In some aspects, the bispecific competitively inhibits binding to Psl of an antibody comprising a heavy chain variable region comprising the amino acid sequence of SEQ ID NO:15 and a light chain variable region comprising the amino acid sequence of SEQ ID NO:16. In some aspects, the bispecific antibody binds to the same epitope of Psl as an antibody comprising a heavy chain variable region comprising the amino acid sequence of SEQ ID NO:15 and a light chain variable region comprising the amino acid sequence of SEQ ID NO:16.

In some aspects, the bispecific antibody comprises a Psl-binding domain comprising a heavy chain CDR1 comprising the amino acid sequence of SEQ ID NO:7, a heavy chain CDR2 comprising the amino acid sequence of SEQ ID NO:8, a heavy chain CDR3 comprising the amino acid sequence of SEQ ID NO:9, a light chain CDR1 comprising the amino acid sequence of SEQ ID NO:10, a light chain CDR2 comprising the amino acid sequence of SEQ ID NO:11, and a light chain CDR3 comprising the amino acid sequence of SEQ ID NO:12. In some aspects, the bispecific antibody comprises a Psl-binding domain comprising a heavy chain variable region comprising the amino acid sequence of SEQ ID NO:15. In some aspects, the bispecific antibody comprises a Psl-binding domain comprising a light chain variable region comprising the amino acid sequence of SEQ ID NO:16. In some aspects, the bispecific antibody comprises a Psl-binding domain comprising a heavy chain variable region comprising the amino acid sequence of SEQ ID NO:15 and a light chain variable region comprising the amino acid sequence of SEQ ID NO:16.

In some aspects, the bispecific antibody comprises a Psl-binding domain with a heavy chain variable region and a light chain variable region on the same polypeptide. In some aspects, the bispecific antibody comprising a Psl-binding domain that is an scFv. In some aspects, the scFv comprises a linker. In some aspects, the linker comprises the amino acid sequence of SEQ ID NO:18. In some aspects, the scFv is in the orientation VH-linker-VL. In some aspects, the scFv comprises the amino acid sequence of SEQ ID NO:17.

In some aspects, bispecific antibody is an IgG antibody. In some aspects, the IgG antibody is an IgG1 antibody.

In some aspects, the bispecific antibody comprises (i) a heavy chain of the formula VH-CH1-H1-L1-S-L2-H2-CH2-CH3, wherein VH is an anti-P. aeruginosa PcrV heavy chain variable domain; CH1 is a heavy chain constant region domain 1; H1 is a first heavy chain hinge region fragment; L1 is a first linker; S is an anti-P. aeruginosa Psl ScFv molecule; L2 is a second linker; H2 is a second heavy chain hinge region fragment; CH2 is a heavy chain constant region domain-2; and CH3 is a heavy chain constant region domain-3; and (ii) a light chain of the formula VL-CL, wherein VL is an anti-P. aeruginosa PcrV light chain variable domain, and CL is an antibody light chain kappa constant region or an antibody light chain lambda region. In some aspects, the VH comprises the amino acid sequence of SEQ ID NO:13. In some aspects, the scFv comprises the amino acid sequence of SEQ ID NO:15 and the amino acid sequence of SEQ ID NO:16. In some aspects, the VL comprises the amino acid sequence of SEQ ID NO:14. In some aspects, the VH comprises the amino acid sequence of SEQ ID NO:13 and the scFv comprises the amino acid sequence of SEQ ID NO:15 and the amino acid sequence of SEQ ID NO:16. In some aspects, the VH comprises the amino acid sequence of SEQ ID NO:13 and the VL comprises the amino acid sequence of SEQ ID NO:14. In some aspects, the scFv comprises the amino acid sequence of SEQ ID NO:15 and the amino acid sequence of SEQ ID NO:16, and the VL comprises the amino acid sequence of SEQ ID NO:14. In some aspects, the VH comprises the amino acid sequence of SEQ ID NO:13, the scFv comprises the amino acid sequence of SEQ ID NO:15 and the amino acid sequence of SEQ ID NO:16, and the VL comprises the amino acid sequence of SEQ ID NO:14. In some aspects, the VH comprises the amino acid sequence of SEQ ID NO:13 and the scFv comprises the amino acid sequence of SEQ ID NO:17. In some aspects, the scFv comprises the amino acid sequence of SEQ ID NO:17 and the VL comprises the amino acid sequence of SEQ ID NO:14. In some aspects, the VH comprises the amino acid sequence of SEQ ID NO:13, the scFv comprises the amino acid sequence of SEQ ID NO:17, and the VL comprises the amino acid sequence of SEQ ID NO:14. In some aspects, CL is an antibody light chain kappa constant region. In some aspects, the bispecific antibody comprises a heavy chain comprising the amino acid sequence of SEQ ID NO:19. In some aspects, the bispecific antibody comprises a light chain comprising the amino acid sequence of SEQ ID NO:20. In some aspects, the bispecific antibody comprises a heavy chain comprising the amino acid sequence of SEQ ID NO:19 and a light chain comprising the amino acid sequence of SEQ ID NO:20.

In some aspects, the bispecific antibody neutralizes cellular intoxication. In some aspects, the bispecific antibody targets Pseudomonas aeruginosa for opsonophagocytic killing. In some aspects, the bispecific antibody prevents cell attachment. In some aspects, the bispecific antibody disrupts biofilm formation. In some aspects, the bispecific antibody inhibits primary colony formation.

In some aspects, the subject is colonized with a Pseudomonas aeruginosa strain comprising a genome comprising a Psl-operon. In some aspects, the subject is colonized with a Pseudomonas aeruginosa strain comprising a genome comprising a PcrV-encoding loci. In some aspects, the subject is human.

In some aspects, the bispecific antibody is administered intravenously. In some aspects, the bispecific antibody is administered subcutaneously.

In some aspects, a method provided herein further comprises administering an antibiotic.

In some aspects provided herein are uses of bispecific antibodies. In some aspects provided herein is a use of a bispecific antibody that specifically binds to Pseudomonas aeruginosa PcrV protein and Psl exopolysaccharide in the preparation of a medicament for treating bronchiectasis in a subject in need thereof. The treating bronchiectasis can comprise any method as provided herein.

In some aspects provided herein are bispecific antibodies for use. In some aspects, provided herein is a bispecific antibody that specifically binds to Pseudomonas aeruginosa PcrV protein and Psl exopolysaccharide for use in treating bronchiectasis in a subject in need thereof. The treating bronchiectasis can comprise any method as provided herein.

### BRIEF DESCRIPTION OF THE DRAWINGS/FIGURES

FIG. 1 shows the target conservation of Psl and PcrV in bronchiectasis (BE)-derived Pseudomonas aeruginosa isolates—derived through WGS (whole genome sequencing). White indicates the presence of the pcrV or psl operon gene, and grey indicates its absence.

FIG. 2 shows the ELISA reactivity of NCFBE-derived Pseudomonas aeruginosa strains 79 and 6 to Cam003 and MEDI3902, indicating expression of Psl in these strains.

FIG. 3 shows immunoblots of NCFBE-derived Pseudomonas aeruginosa strains using the anti-PcrV antibody V2L2-MD and an anti-PcrV polyclonal IgG. (Strains 6077 and 6206 were used as positive controls).

FIG. 4A shows the anti-cytotoxic activity of MEDI3902 and control IgG against representative ExoU-producing P. aeruginosa non-cystic fibrosis bronchiectasis (NCFBE) isolates (NCFBE strains 84 and 55). The x-axis shows the log concentration of mAb used (nM).

FIG. 4B shows an in vitro opsonophagocytosis (OPK) activity of MEDI3902 and control IgG against NCFBE-derived P. aeruginosa strain 6. The x-axis shows the log concentration of mAb used (nM).

FIG. 5 shows the effect of MEDI3902 on bronchiectasis patient peripheral-blood neutrophil OPK of P. aeruginosa, as well as the effect on peripheral-blood neutrophil OPK against P. aeruginosa of neutrophils derived from healthy controls and matched controls. Neutrophil activities after exposure to IgG control are also shown.

FIGS. 6A and 6B show endogenous anti-Psl and anti-PcrV serum titres in bronchiectasis patients and matched controls. Measurement of antibody titres was performed in serum samples from bronchiectasis patients, stratified into those with chronic P. aeruginosa airway infection (chronic PA) and those without recent recorded infection (no known PA) and age- and sex-matched controls. FIG. 6A—ELISA plates coated with (left) wild-type (WT) PAO1 or (right) pslA-deficient (ΔpslA) PAO1 were used to determine serum anti-Psl antibody titres. Serum was added in a dilution series from 1:200-1:437400 to determine endpoint titres, and PAO1 binding detected using anti-human HRP-conjugated antibody. Endpoint titres were those which demonstrated a difference in absorbance values between two consecutive dilutions less than the value of the blank, and were considered to be anti-Psl specific if the titre was lowered in the ΔPsl-PAO1-coated wells in right panel. Data points represent individuals identified with endogenous anti-Psl antibodies. FIG. 6B—ELISA plates coated with recombinant human PcrV protein were used to determine anti-PcrV titres, and endpoint titres were selected as in FIG. 6A. Serum was added in a dilution series from 1:100-1:72900. Lines indicate the group median.

FIGS. 7A and 7B show bronchiectasis patient endogenous anti-Psl antibody function and effect on MEDI3902 activity. FIG. 7A—Isolated peripheral blood neutrophils were incubated with luminescent P. aeruginosa (PAO1), either alone or with serum at the indicated dilutions. In the left panel, wild-type (WT) PAO1 was utilized to establish effects on neutrophil-mediated bacterial killing, and in the right panel, Psl-deficient (ΔpslA) PAO1 was utilized to determine the specific contribution of anti-Psl antibodies. FIG. 7B—To evaluate whether endogenous anti-Psl antibodies compete with MEDI3902, the OPK assay was repeated with MEDI3902 alone at the indicated concentrations or spiked into serum diluted 1:100 using WT PAO1 (left panel) or PAO1ΔpslA (right panel). OPK was measured after 2 h by quantitating luminescence. % increase in neutrophil OPK was calculated relative to that of neutrophils incubated with PAO1 without antibody addition. Data represents mean±SD for MEDI3902 alone, or average of duplicate wells for serum experiments.

FIGS. 8A and 8B show the effect of bronchiectasis patient serum containing endogenous anti-PcrV antibodies on P. aeruginosa cytotoxicity and MEDI3902 activity. FIG. 8A—Bronchiectasis patient serum containing endogenous anti-PcrV antibodies as detected by ELISA was added to A549 epithelial cells at the indicated dilutions, together with a cytotoxic P. aeruginosa strain (6077) to assess anti-PcrV functionality. Cell lysis was determined by measurement of lactate dehydrogenase after 3 h incubation. FIG. 8B—Capacity to abrogate cytotoxic effects of P. aeruginosa on epithelial cells was determined as in FIG. 8A for either MEDI3902 alone (n=3 separate experimental runs) or serum samples used in FIG. 8A at 1:100 dilution were spiked with MEDI3902 at the indicated concentrations. Data represents mean±SD for MEDI3902 alone, or the average of duplicate wells for serum experiments.

FIG. 9 provides a flow chart of a clinical trial demonstrating the efficacy of MEDI3902 in bronchiectasis patients.

### DETAILED DESCRIPTION OF THE INVENTION

### I. Definitions

The headings provided herein are not limitations of the various aspects or aspects of the disclosure, which can be had by reference to the specification as a whole. Accordingly, the terms defined immediately below are more fully defined by reference to the specification in its entirety.

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

Units, prefixes, and symbols are denoted in their Système International de Unites (SI) accepted form. Numeric ranges are inclusive of the numbers defining the range.

Unless otherwise indicated, amino acid sequences are written left to right in amino to carboxy orientation.

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

As used herein, the terms “antibody” and “immunoglobulin” are used interchangeably and refer to an antibody molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. The term “antibody” encompasses monoclonal antibodies, chimeric antibodies, humanized antibodies, human antibodies, bispecific antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1, and IgA2), based on the identity of their heavy-chain constant domains referred to as alpha, delta, epsilon, gamma, and mu, respectively. The different classes of antibodies have different and well known subunit structures and three-dimensional configurations. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The term “antibody fragment” refers to a portion of an antibody. An “antigen-binding fragment” of an antibody refers to a portion of an antibody that binds to an antigen. An antigen-binding fragment of an antibody can comprise the antigenic determining regions of an antibody (e.g., the complementarity determining regions (CDRs)). Examples of antigen-binding fragments of antibodies include, but are not limited to Fab, Fab′, F(ab′)2, and Fv fragments, linear antibodies, and single chain antibodies. An antigen-binding fragment of an antibody can be monovalent or multi-valent (e.g., bivalent). An antigen-binding fragment of an antibody can be monospecific or multi-specific (e.g., bispecific.) An antigen-binding fragment of an antibody can be derived from any animal species, such as rodents (e.g., mouse, rat, or hamster) and humans or can be artificially produced.

An “antigen-binding domain” or “antigen-binding region” refers to a monovalent portion of an antibody that binds to an antigen. An “antigen-binding domain” can comprise the antigenic determining regions of an antibody (e.g., the complementarity determining regions (CDRs)). An antibody or antigen-binding fragment thereof (including mono-specific and multi-specific (e.g., bispecific) antibodies or antigen-binding fragments thereof can comprise an antigen-binding domain.

Antibody fragments including single-chain antibodies can comprise the variable region(s) alone or in combination with the entirety or a portion of the following: hinge region, CH1, CH2, and CH3 domains.

Also included are antigen-binding fragments also comprising any combination of variable region(s) with a hinge region, CH1, CH2, and CH3 domains.

Antibodies, or antigen-binding fragments thereof disclosed herein can be from any animal origin including birds and mammals. The antibodies can be human, murine, donkey, rabbit, goat, guinea pig, camel, llama, horse, or chicken antibodies.

Light chains are classified as either kappa or lambda (x, k). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen binding site present at the end of each arm of the Y. More specifically, the antigen binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

Single chain Fvs (scFv) molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

By “specifically binds,” it is generally meant that a binding molecule, e.g., a bispecific antibody or fragment, variant, or derivative thereof binds to an epitope via an antigen binding domain, and that the binding entails some complementarity between an antigen binding domain and the epitope. A binding molecule as provided herein can contain one, two, three, four, or more binding domains that can be the same or different, and that can bind to the same epitope, or to two or more different epitopes. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” may be deemed to have a higher specificity for a given epitope than binding molecule “B,” or binding molecule “A” may be said to bind to epitope “C” with a higher specificity than it has for related epitope “D.”

An antibody that “binds to the same epitope” as a reference antibody refers to an antibody that contacts the same amino acid and/or sugar residues as the reference antibody. The ability of an antibody to bind to the same epitope as a reference antibody can be determined using peptide scanning mutagenesis or high throughput alanine scanning mutagenesis.

An antibody is said to “competitively inhibit” binding of a reference antibody to a given epitope if it preferentially binds to that epitope or an overlapping epitope such that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The term “bispecific antibody” as used herein refer to an antibody that has binding domains specific for two different antigens or epitopes within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)).

As used herein, the term “MEDI3902” refers to a bispecific antibody comprising a heavy chain with the amino acid sequence of SEQ ID NO:19 and a light chain with the amino acid sequence of SEQ ID NO:20. MEDI3902 is also known as Gremubamab.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

Administration “in combination with” one or more further therapeutic agents includes simultaneous (concurrent) or consecutive administration in any order.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder.

The term “airway neutrophilia” refers to an accumulation of neutrophils in the airspace of the lungs.

The term “sputum neutrophilia” refers to the presence of neutrophils in the sputum of a subject. In some aspects, the neutrophils in the sputum of a subject in need of treatment, e.g., a subject with bronchiectasis, are increased relative to neutrophils in the sputum of healthy controls.

By “subject” or “individual” or “animal” or “patient” or “mammal,” is meant any subject, e.g., a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

### II. Methods and Uses of Bispecific Antibodies in the Treatment of Bronchiectasis

As demonstrated herein, bispecific antibodies that specifically bind to Pseudomonas aeruginosa Psl and PcrV are useful in treating subjects with bronchiectasis. Accordingly, provided herein are methods of treatment using such bispecific antibodies, uses of such bispecific antibodies in the preparation of medicaments, and bispecific antibodies for use in treatments.

In some aspects, provided herein are methods of treating bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of improving pre-bronchodilator forced expiratory volume 1 (FEV₁) in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of reducing Pseudomonas aeruginosa load in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of reducing bronchiectasis exacerbations in a subject with bronchiectasis (e.g., non-cystic fibrosis bronchiectasis). The methods can, for example, reduce bronchiectasis exacerbations requiring hospitalization and/or reduce bronchiectasis exacerbations requiring antibiotics The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of reducing the need for intravenous antibiotics in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of stabilizing lung function in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of improving cough frequency and/or intensity in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of decreasing bronchiectasis symptoms in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of improving the quality of life in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of eradicating Pseudomonas aeruginosa in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of inducing sustained Pseudomonas aeruginosa suppression in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

In some aspects, provided herein are methods of reducing the risk of bronchiectasis progression related to Pseudomonas aeruginosa in a subject with bronchiectasis (e.g., non-cystic fibrosis bronchiectasis). The methods can comprise administering to the subject a bispecific antibody that specifically binds to Pseudomonas aeruginosa PcrV protein and Psl exopolysaccharide (e.g., MEDI3902). The subject can be, e.g., a subject that is colonized with Pseudomonas aeruginosa.

A bronchiectasis suitable for treatment in accordance with the methods and uses provided herein can be non-cystic fibrosis bronchiectasis. In some aspects, the non-cystic fibrosis bronchiectasis was confirmed by chest computed tomography (CT) demonstrating bronchiectasis affecting 1 or more lobes in a subject.

A subject with bronchiectasis (e.g., non-cystic fibrosis bronchiectasis) suitable for treatment in accordance with the methods and uses provided herein can be a subject that is colonized with Pseudomonas aeruginosa. Subjects with Pseudomonas aeruginosa colonization can be identified, e.g., using routine sputum culture. In some aspects, the subject is colonized with a Pseudomonas aeruginosa strain comprising a genome comprising a Psl-operon. In some aspects, the subject is colonized with a Pseudomonas aeruginosa strain comprising a genome comprising a PcrV-encoding loci.

In some aspects, the subject is chronically infected with Pseudomonas aeruginosa. As used herein, a subject who is “chronically infected” refers to a subject colonized with at least two (2) isolates of Pseudomonas aeruginosa, concurrently or sequentially, while clinically stable over a year.

A subject with bronchiectasis (e.g., non-cystic fibrosis bronchiectasis) suitable for treatment in accordance with the methods and uses provided herein can be a subject with airway neutrophilia and/or sputum neutrophilia.

A subject with bronchiectasis (e.g., non-cystic fibrosis bronchiectasis) suitable for treatment in accordance with the methods and uses provided herein can be a subject with a history of at least two (2) moderate to severe bronchiectasis exacerbations per year requiring antibiotics.

A subject with bronchiectasis (e.g., non-cystic fibrosis bronchiectasis) suitable for treatment in accordance with the methods and uses provided herein can be a subject with a history of at least one (1) exacerbation requiring hospital care.

A subject with bronchiectasis (e.g., non-cystic fibrosis bronchiectasis) suitable for treatment in accordance with the methods and uses provided herein can be a subject on long term nebulized antibiotics.

A subject with bronchiectasis (e.g., non-cystic fibrosis bronchiectasis) suitable for treatment in accordance with the methods and uses provided herein can be a subject with chronic obstructive pulmonary disease (COPD).

A subject with bronchiectasis (e.g., non-cystic fibrosis bronchiectasis) suitable for treatment in accordance with the methods and uses provided herein can be a human subject.

As provided herein, bronchiectasis (e.g., non-cystic fibrosis bronchiectasis) of any cause can be treated in accordance with the methods and uses provided herein. For example, in some aspects, the bronchiectasis (e.g., non-cystic fibrosis bronchiectasis) was caused by hypogammaglobulinemia. In some aspects, the bronchiectasis (e.g., non-cystic fibrosis bronchiectasis) was caused by common variable immunodeficiency. In some aspects, the bronchiectasis (e.g., non-cystic fibrosis bronchiectasis) was caused by alpha-1-antitrypsin deficiency.

The methods and uses provided herein can comprise administering a bispecific antibody that specifically binds to Pseudomonas aeruginosa PcrV protein and Psl exopolysaccharide (e.g., MEDI3902) to a subject. The administration can be intravenous administration. The administration can be subcutaneous administration.

The methods and uses provided herein are effective in treating bronchiectasis (e.g., non-cystic fibrosis bronchiectasis). In some aspects of the methods and uses provided herein, administration of a bispecific antibody that specifically binds to Pseudomonas aeruginosa PcrV protein and Psl exopolysaccharide (e.g., MEDI3902) reduces Pseudomonas aeruginosa in sputum cultures obtained from the subject, e.g., as compared to the Pseudomonas aeruginosa in sputum cultures obtained from the subject prior to the administration. The reduction can occur, e.g., within 12 weeks of the first administration of the bispecific antibody, within 8 weeks of the first administration of the bispecific antibody, or within 4 weeks of the first administration of the bispecific antibody. In some aspects of the methods and uses provided herein, administration of a bispecific antibody that specifically binds to Pseudomonas aeruginosa PcrV protein and Psl exopolysaccharide (e.g., MEDI3902) decreases antibiotic usage by the subject, e.g., as compared to the subject's usage prior to the administration. In some aspects of the methods and uses provided herein, administration of a bispecific antibody that specifically binds to Pseudomonas aeruginosa PcrV protein and Psl exopolysaccharide (e.g., MEDI3902) eradicates Pseudomonas aeruginosa in the subject.

The methods and uses provided herein can comprise administering a bispecific antibody that specifically binds to Pseudomonas aeruginosa PcrV protein and Psl exopolysaccharide (e.g., MEDI3902) to a subject in combination with an antibiotic. In some aspects, the methods and uses provided herein comprise administering a bispecific antibody that specifically binds to Pseudomonas aeruginosa PcrV protein and Psl exopolysaccharide (e.g., MEDI3902) to a subject in combination with an aminoglycoside, ticarcillin, a ureidopenicillin, ciprofloxacin, cefepime, gentamicin, amikacin, tobramycin, ceftazidime, aztreonam, cefotaxime, meropenem, polymyxin b, or any combination thereof.

### III. Bispecific Antibodies

The methods and uses provided herein utilize bispecific antibodies that specifically bind to Pseudomonas aeruginosa Psl and PcrV. The bispecific antibodies comprise a Psl-binding domain and a PcrV-binding domain. Exemplary bispecific antibodies for use in the methods provided herein are disclosed, e.g., in PCT Publication Nos. WO 2013/070615, WO 2014/074528, and WO 2015/171504, the disclosures of which are incorporated by reference herein in their entireties.

Exemplary sequences of a bispecific antibody for use in the methods provided herein are presented in Table 1.

TABLE 1
Bispecific Antibody Sequences

| SEQ | | |
|---|---|---|
| ID | | |
| NO: | Description | Sequence |
| 1 | Anti- | SYAMN |
| | PcrV | |
| | HCDR1 | |
| 2 | Anti- | AITMSGITAYYTDDVKG |
| | PcrV | |
| | HCDR2 | |
| 3 | Anti- | EEFLPGTHYYYGMDV |
| | PcrV | |
| | HCDR3 | |
| 4 | Anti- | RASQGIRNDLG |
| | PcrV | |
| | LCDR1 | |
| 5 | Anti- | SASTLQS |
| | PcrV | |
| | LCDR2 | |
| 6 | Anti- | LQDYNYPWT |
| | PcrV | |
| | LCDR3 | |
| 7 | Anti-Psl | PYYWT |
| | HCDR1 | |
| 8 | Anti-Psl | YIHSSGYTDYNPSLKS |
| | HCDR2 | |
| 9 | Anti-Psl | ADWDRLRALDI |
| | HCDR3 | |
| 10 | Anti-Psl | RASQSIRSHLN |
| | LCDR1 | |
| 11 | Anti-Psl | GASNLQS |
| | LCDR2 | |
| 12 | Anti-Psl | QQSTGAWNW |
| | LCDR3 | |
| 13 | Anti- | EVQLLESGGGLVQPGGSLRLSCAASGFTFSSYAMNWVRQAPGK |
| | PcrV VH | GLEWVSAITMSGITAYYTDDVKGRFTISRDNSKNTLYLQMNSLR |
| | | AEDTAVYYCAKEEFLPGTHYYYGMDVWGQGTTVTVSS |
| 14 | Anti- | AIQMTQSPSSLSASVGDRVTITCRASQGIRNDLGWYQQKPGKAP |
| | PcrV VL | KLLIYSASTLQSGVPSRFSGSGSGTDFTLTISSLQPEDFATYYCLQ |
| | | DYNYPWTFGQGTKVEIK |
| 15 | Anti-Psl | QVQLQESGPGLVKPSETLSLTCTVSGGSISPYYWTWIRQPPGKCL |
| | VH | ELIGYIHSSGYTDYNPSLKSRVTISGDTSKKQFSLKLSSVTAADTA |
| | | VYYCARADWDRLRALDIWGQGTMVTVSS |
| 16 | Anti-Psl | DIQLTQSPSSLSASVGDRVTITCRASQSIRSHLNWYQQKPGKAPK |
| | VL | LLIYGASNLQSGVPSRFSGSGSGTDFTLTISSLQPEDFATYYCQQS |
| | | TGAWNWFGCGTKVEIK |
| 17 | Anti-Psl | QVQLQESGPGLVKPSETLSLTCTVSGGSISPYYWTWIRQPPGKCL |
| | scFv | ELIGYIHSSGYTDYNPSLKSRVTISGDTSKKQFSLKLSSVTAADTA |
| | | VYYCARADWDRLRALDIWGQGTMVTVSSGGGGSGGGGSGGGG |
| | | SGGGGSDIQLTQSPSSLSASVGDRVTITCRASQSIRSHLNWYQQK |
| | | PGKAPKLLIYGASNLQSGVPSRFSGSGSGTDFTLTISSLQPEDFAT |
| | | YYCQQSTGAWNWFGCGTKVEIK |
| 18 | Anti-Psl | GGGGSGGGGSGGGGSGGGGS |
| | scFv | |
| | linker | |
| 19 | MEDI- | EVQLLESGGGLVQPGGSLRLSCAASGFTFSSYAMNWVRQAPGK |
| | 3902 | GLEWVSAITMSGITAYYTDDVKGRFTISRDNSKNTLYLQMNSLR |
| | Heavy | AEDTAVYYCAKEEFLPGTHYYYGMDVWGQGTTVTVSSASTKG |
| | | PSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCGGGGSGGGGSQVQLQESGPGLVKPSETLSLTCTVSG |
| | | GSISPYYWTWIRQPPGKCLELIGYIHSSGYTDYNPSLKSRVTISGD |
| | | TSKKQFSLKLSSVTAADTAVYYCARADWDRLRALDIWGQGTM |
| | | VTVSSGGGGSGGGGSGGGGSGGGGSDIQLTQSPSSLSASVGDRV |
| | | TITCRASQSIRSHLNWYQQKPGKAPKLLIYGASNLQSGVPSRFSG |
| | | SGSGTDFTLTISSLQPEDFATYYCQQSTGAWNWFGCGTKVEIKG |
| | | GGGSGGGGSDKTHTCPPCPAPELLGGPSVFLFPPKPKDTLMISRT |
| | | PEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 20 | MEDI- | AIQMTQSPSSLSASVGDRVTITCRASQGIRNDLGWYQQKPGKAP |
| | 3902 | KLLIYSASTLQSGVPSRFSGSGSGTDFTLTISSLQPEDFATYYCLQ |
| | Light | DYNYPWTFGQGTKVEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYACEVTHQGLSSPVTKSFNRGEC |
| 21 | CH1 | ASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSN |
| | | TKVDKRV |
| 22 | H1 | EPKSC |
| 23 | H2 | DKTHTCPPCP |
| 24 | CL | RTVAAPSVFIFPPSDEQLKSGTASVVCLLNNFYPREAKVQWKVD |
| | | NALQSGNSQESVTEQDSKDSTYSLSSTLTLSKADYEKHKVYACE |
| | | VTHQGLSSPVTKSFNRGEC |
| 25 | CH2- | APELLGGPSVFLFPPKPKDTLMISRTPEVTCVVVDVSHEDPEVKFN |
| | CH3 | WYVDGVEVHNAKTKPREEQYNSTYRVVSVLTVLHQDWLNGKE |
| | | YKCKVSNKALPAPIEKTISKAKGQPREPQVYTLPPSREEMTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPGK |
| 26 | Linker | [GGGGS]n, where n is 0, 1, 2, 3, 4, or 5 |
| 27 | Linker | [GGGG]n, where n is 0, 1, 2, 3, 4, or 5 |
| 28 | L1 | [GGGGS]n, where n is 2 |
| 29 | L2 | [GGGGS]n, where n is 4 |

A bispecific antibody for the methods and uses provided herein can comprise an antigen-binding domain that specifically binds to P. aeruginosa PcrV and competitively inhibits binding to PcrV of an antibody comprising a heavy chain variable region comprising the amino acid sequence of SEQ TD NO:13 and a light chain variable region comprising the amino acid sequence of SEQ ID NO:14. In some aspects, a bispecific antibody for the methods and uses provided herein comprises an antigen-binding domain that specifically binds to P. aeruginosa PcrV and binds to the same epitope of PcrV as an antibody comprising a heavy chain variable region comprising the amino acid sequence of SEQ ID NO:13 and a light chain variable region comprising the amino acid sequence of SEQ ID NO:14.

In some aspects, a bispecific antibody for the methods and uses provided herein comprises an antigen-binding domain that specifically binds to P. aeruginosa PcrV and comprises (i) a heavy chain CDR1, CDR2, and CDR3 comprising the amino acid sequences of the heavy chain CDR1, CDR2, and CDR3 sequences in SEQ ID NO:13 (e.g., the Kabat-defined, AbM-defined, or Chothia-defined CDRs) and (ii) a light chain CDR1, CDR2, and CDR3 comprising the amino acid sequences of the heavy chain CDR1, CDR2, and CDR3 sequences in SEQ ID NO:14 (e.g., the Kabat-defined, AbM-defined, or Chothia-defined CDRs).

In some aspects, a bispecific antibody for the methods and uses provided herein comprises an antigen-binding domain that specifically binds to P. aeruginosa PcrV and comprises a heavy chain CDR1 comprising the amino acid sequence of SEQ ID NO:1, a heavy chain CDR2 comprising the amino acid sequence of SEQ ID NO:2, a heavy chain CDR3 comprising the amino acid sequence of SEQ ID NO:3, a light chain CDR1 comprising the amino acid sequence of SEQ ID NO:4, a light chain CDR2 comprising the amino acid sequence of SEQ ID NO:5, and a light chain CDR3 comprising the amino acid sequence of SEQ ID NO:6. In some aspects, a bispecific antibody for the methods and uses provided herein comprises an antigen-binding domain that specifically binds to P. aeruginosa PcrV and comprises a heavy chain variable region comprising the amino acid sequence of SEQ ID NO:13. In some aspects, a bispecific antibody for the methods and uses provided herein comprises an antigen-binding domain that specifically binds to P. aeruginosa PcrV and comprises a light chain variable region comprising the amino acid sequence of SEQ ID NO:14. In some aspects, a bispecific antibody for the methods and uses provided herein comprises an antigen-binding domain that specifically binds to P. aeruginosa PcrV and comprises a heavy chain variable region comprising the amino acid sequence of SEQ ID NO:13 and a light chain variable region comprising the amino acid sequence of SEQ ID NO:14.

In some aspects, a bispecific antibody for the methods and uses provided herein comprises a PcrV-binding domain with a heavy chain variable region and a light chain variable region on separate polypeptides.

In some aspects, a bispecific antibody for the methods and uses provided herein comprises an antigen binding domain that specifically binds to P. aeruginosa Psl and competitively inhibits binding to Psl of an antibody comprising a heavy chain variable region comprising the amino acid sequence of SEQ ID NO:15 and a light chain variable region comprising the amino acid sequence of SEQ ID NO:16. In some aspects, a bispecific antibody for the methods and uses provided herein comprises an antigen-binding domain that specifically binds to P. aeruginosa Psl and binds to the same epitope of Psl as an antibody comprising a heavy chain variable region comprising the amino acid sequence of SEQ ID NO:15 and a light chain variable region comprising the amino acid sequence of SEQ ID NO:16.

In some aspects, a bispecific antibody for the methods and uses provided herein comprises an antigen-binding domain that specifically binds to P. aeruginosa Psl and comprises (i) a heavy chain CDR1, CDR2, and CDR3 comprising the amino acid sequences of the heavy chain CDR1, CDR2, and CDR3 sequences in SEQ ID NO:15 (e.g., the Kabat-defined, AbM-defined, or Chothia-defined CDRs) and (ii) a light chain CDR1, CDR2, and CDR3 comprising the amino acid sequences of the heavy chain CDR1, CDR2, and CDR3 sequences in SEQ ID NO:16 (e.g., the Kabat-defined, AbM-defined, or Chothia-defined CDRs).

In some aspects, a bispecific antibody for the methods and uses provided herein comprises an antigen-binding domain that specifically binds to P. aeruginosa Psl and comprises a heavy chain CDR1 comprising the amino acid sequence of SEQ ID NO:7, a heavy chain CDR2 comprising the amino acid sequence of SEQ ID NO:8, a heavy chain CDR3 comprising the amino acid sequence of SEQ ID NO:9, a light chain CDR1 comprising the amino acid sequence of SEQ ID NO:10, a light chain CDR2 comprising the amino acid sequence of SEQ ID NO:11, and a light chain CDR3 comprising the amino acid sequence of SEQ ID NO:12. In some aspects, a bispecific antibody for the methods and uses provided herein comprises an antigen-binding domain that specifically binds to P. aeruginosa Psl and comprises a heavy chain variable region comprising the amino acid sequence of SEQ ID NO:15. In some aspects, a bispecific antibody for the methods and uses provided herein comprises an antigen-binding domain that specifically binds to P. aeruginosa Psl and comprises a light chain variable region comprising the amino acid sequence of SEQ ID NO:16. In some aspects, a bispecific antibody for the methods and uses provided herein comprises an antigen-binding domain that specifically binds to P. aeruginosa Psl and comprises a heavy chain variable region comprising the amino acid sequence of SEQ ID NO:15 and a light chain variable region comprising the amino acid sequence of SEQ ID NO:16.

In some aspects, a bispecific antibody for the methods and uses provided herein comprises an antigen-binding domain that specifically binds to P. aeruginosa Psl and comprises a heavy chain variable region and a light chain variable region on the same polypeptide. In some aspects, the bispecific antibody comprises a Psl-binding domain that is an scFv. The scFv can comprise a linker. The linker can be, for example, a glycine-rich linker or a glycine-serine linker. In some aspects, the linker comprises the amino acid sequence of SEQ ID NO:18. In some aspects, the scFv is in the orientation VH-linker-VL. In some aspects, the scFv is in the orientation VL-linker-VL. In some aspects, the scFv comprises the amino acid sequence of SEQ ID NO:17.

In some aspects, a bispecific antibody for the methods and uses provided herein is an IgG antibody. The IgG antibody can be, for example, an IgG1 antibody.

In some aspects, a bispecific antibody for the methods and uses provided herein comprises (i) a heavy chain of the formula VH-CH1-H1-L1-S-L2-H2-CH2-CH3, wherein VH is an anti-P. aeruginosa PcrV heavy chain variable domain; CH1 is a heavy chain constant region domain 1; H1 is a first heavy chain hinge region fragment; L1 is a first linker; S is an anti-P. aeruginosa Psl ScFv molecule; L2 is a second linker; H2 is a second heavy chain hinge region fragment; CH2 is a heavy chain constant region domain-2; and CH3 is a heavy chain constant region domain-3; and (ii) a light chain of the formula VL-CL, wherein VL is an anti-P. aeruginosa PcrV light chain variable domain, and CL is an antibody light chain kappa constant region or an antibody light chain lambda region. In some aspects, CL is an antibody light chain kappa constant region. In some aspects, the VH comprises the amino acid sequence of SEQ ID NO:13, and the VL comprises the amino acid sequence of SEQ ID NO:14. In some aspects, the scFv comprises the amino acid sequence of SEQ ID NO:15 and the amino acid sequence of SEQ ID NO:16. In some aspects, the VH comprises the amino acid sequence of SEQ ID NO:13, the VL comprises the amino acid sequence of SEQ ID NO:14, the scFv comprises the amino acid sequence of SEQ ID NO:15 and the amino acid sequence of SEQ ID NO:16. In some aspects, the scFv comprises the amino acid sequence of SEQ ID NO:17. In some aspects, the VH comprises the amino acid sequence of SEQ ID NO:13, the VL comprises the amino acid sequence of SEQ ID NO:14, the scFv comprises the amino acid sequence of SEQ ID NO:17. In some aspects, CH1 comprises the amino acid sequence of SEQ ID NO:21. In some aspects, L1 and L2 can be the same or different, and can independently comprise (a) [GGGGS]n, where n is 0, 1, 2, 3, 4, or 5 (SEQ ID NO:26), (b) [GGGG]n, where n is 0, 1, 2, 3, 4, or 5 (SEQ ID NO:27), or a combination of (a) and (b). In some aspects, H1 comprises the amino acid sequence EPKSC (SEQ ID NO:22). In some aspects, L1 comprises [GGGGS]n, where n is 2 (SEQ ID NO:28). In some aspects, L2 comprises [GGGGS]n, where n is 2 (SEQ ID NO: 28) or n is 4 (SEQ ID NO:29). In some aspects, H2 comprises the amino acid sequence DKTHTCPPCP (SEQ ID NO:23). In some aspects CH2-CH3 comprises the amino acid sequence of SEQ ID NO:25. In some aspects, CL comprises the amino acid sequence of SEQ ID NO:24.

In some aspects, a bispecific antibody for the methods and uses provided herein comprises a polypeptide comprising the amino acid sequence of SEQ ID NO:19. In some aspects, a bispecific antibody for the methods and uses provided herein comprises a polypeptide comprising the amino acid sequence of SEQ ID NO:20. In some aspects, a bispecific antibody for the methods and uses provided herein comprises a polypeptide comprising the amino acid sequence of SEQ ID NO:19 and a polypeptide comprising the amino acid sequence of SEQ ID NO:20.

In some aspects, a bispecific antibody for the methods and uses provided herein neutralizes cellular intoxication. In some aspects, a bispecific antibody for the methods and uses provided herein targets Pseudomonas aeruginosa for opsonophagocytic killing (OPK). Methods of assessing neutralization of cellular intoxication and/or assessing OPK are known in the art and provided herein, e.g., in Example 3. In some aspects, a bispecific antibody for the methods and uses provided herein prevents cell attachment, e.g., prevents attachment of Pseudomonas aeruginosa to host cells. Methods of assessing prevention of attachment are known in the art and provided, e.g., in WO 2013/070615, which is herein incorporated by reference in its entirety. In some aspects, a bispecific antibody for the methods and uses provided herein disrupts biofilm formation. In some aspects, a bispecific antibody for the methods and uses provided herein inhibits primary colony formation.

Anti-Pseudomonas aeruginosa Psl and PcrV bispecific antibody can be for use in the preparation for a medicament for treating bronchiectasis (e.g., non-cystic fibrosis bronchiectasis).

### IV. Pharmaceutical Compositions

Pharmaceutical compositions used in this disclosure include anti-Pseudomonas aeruginosa Psl and PcrV bispecific antibodies and pharmaceutically acceptable carriers well known to those of ordinary skill in the art. Preparations for parenteral administration include sterile aqueous or non-aqueous solutions, suspensions, and emulsions.

The route of administration of an anti-Pseudomonas aeruginosa Psl and PcrV bispecific antibody can be, for example, parenteral. The term parenteral as used herein includes, e.g., intravenous and subcutaneous administration. Accordingly, a pharmaceutical composition comprising an anti-Pseudomonas aeruginosa Psl and PcrV bispecific antibody can be formulated for intravenous administration. In some aspects, a pharmaceutical composition comprising an anti-Pseudomonas aeruginosa Psl and PcrV bispecific antibody can be formulated for subcutaneous administration. A suitable form for administration would be a solution for injection.

Pharmaceutical compositions comprising anti-Pseudomonas aeruginosa Psl and PcrV bispecific antibodies can be administered and/or formulated, e.g., for the treatment of bronchiectasis (e.g., non-cystic fibrosis bronchiectasis).

As provided herein, pharmaceutical compositions comprising anti-Pseudomonas aeruginosa Psl and PcrV bispecific antibodies can be formulated for administration in combination with an antibiotic. In some aspects, the bispecific antibodies are formulated for administration in combination with an aminoglycoside, ticarcillin, a ureidopenicillin, ciprofloxacin, cefepime, gentamicin, amikacin, tobramycin, ceftazidime, aztreonam, cefotaxime, meropenem, polymyxin b, or any combination thereof.

### EXAMPLES

### Materials and Methods for Examples 1-4

### Patients

Patients with bronchiectasis were enrolled from a specialist bronchiectasis clinic at Ninewells Hospital in Dundee, UK. The inclusion criteria for bronchiectasis patients were: age >18 years, bronchiectasis confirmed by CT scan with associated respiratory symptoms, and ability to give informed consent. Healthy and age- and sex-matched control subjects were also enrolled from Ninewells Hospital; inclusion criteria were age >18 years and ability to give informed consent, participants were excluded if they had active infection or inflammatory diseases.

P. aeruginosa Bronchiectasis Patient Isolates

P. aeruginosa clinical isolates from sputum of 100 individuals with bronchiectasis were utilized for whole genome sequences (WGS) as described previously (Tabor et al., the Journal of Infectious Diseases, 2018, 218:1983-1994—DOI: 10.1093/infdis/jiy438). For initial culture, frozen stored patient isolates were inoculated into lysogeny broth in 96-well Corning P P 1.2 ml sterile cluster tubes (Sigma; CLS4411) and incubated for approximately 24 hours at 37° C. with orbital shaking (200 rpm). Plates were subsequently centrifuged (5000 g, 15 min), and supernatant was removed. Plates containing bacterial cell pellets were frozen at −80° C. until thawing for DNA extraction.

### Whole-Genome Sequencing and Genetic Analysis

Whole genome sequencing and genetic analysis was carried out as described in DOI: 10.1093/infdis/jiy438. In short, DNA was purified from bacterial cultures via bead beating followed by extraction using a QIAamp DNA Mini kit (QIAGEN). Sequencing libraries were prepared by Covaris mechanical shearing followed by a NEBNext Ultra DNA Library Prep Kit (New England BioLabs). Sequencing was performed via NovaSeq 2×250 sequencing runs (Illumina).

Sequence data was quality-trimmed via BBDuk (Bushnell B. http://sourceforge.net/projects/bbmap/), de-novo assembled via SPAdes (Bankevich et al., J Comput Biol., 2012, 19:455-477—DOI: 10.1089/cmb.2012.0021) and annotated using Prokka (Seeman, Bioinformatics, 2014, 30:2068-2069—DOI: 10.1093/bioinformatics/btu153). Multilocus sequence typing (MLST) and Psl operon gene content assessment was performed using SRST2 (Inouye et al., Genome Medicine, 2014, 6:90—DOI: 10.1186/s13073-014-0090-6) with a minimum cutoff of 20-fold coverage for gene presence calling. PcrV gene sequences were assembled de novo (Tovchigrechko et al. DOI:10.5281/zenodo.3619527. Accessed 2020/01/21, 2020), translated into protein sequences, and amino acid substitutions were identified using PAO1 NC_002516 pcrV as a reference sequence. Core-genome multi-alignments were performed using Parsnp and the PAO1 reference NC_002516 (Treangen et al., Genome Biology, 2014, 15:524—DOI: 10.1186/s13059-014-0524-x). Phylogenetic trees were visualized and annotated using ITOL, version 3 (Letunic and Bork, Nucleic Acids Research, 2016, 44:W242-W245—DOI: 10.1093/nar/gkw290).

### Endogenous Anti-Psl and Anti-PcrV Antibody Levels in Serum

End-point antibody titres were determined by enzyme-linked immunosorbent assay (ELISA) as described in (Thaden et al., The Journal of Infectious Diseases, 2016, 213:640-648—DOI: 10.1093/infdis/jiv436).

### Human Neutrophil Isolation and Opsonophagocytic Killing (OPK) Assay

Venous blood (20 ml) with EDTA anticoagulant was used for primary neutrophil isolation within 2 hours of venepuncture. Peripheral blood neutrophils were isolated using EasySep™ Direct Human Neutrophil Isolation Kit (StemCell, Cat No. 19666) as per the manufacturer's instructions. Briefly, 50 μl antibody selection cocktail and 50 μl RapidSpheres were added per ml of whole blood. EasySep™ Easy50 magnet was used for immunomagnetic separation. After incubation, RapidSpheres were added once again and blood incubated for a further two separation cycles. Isolated cells were quantitated using a haemocytometer, washed in DPBS and pelleted at 300 g, 6 min. Neutrophils were resuspended in OPK buffer (phenol-red-free RPMI 1640 [Gibco, Cat no. 10363083] with 1% BSA) at 20×106 cells/ml for use.

Neutrophil OPK of P. aeruginosa was measured as previously described (DOI: 10.1093/infdis/jiv436), with modifications described herein. To test the effect of MEDI3902 on primary neutrophil OPK activity, an antibody dilution series from 40 to 0.04 μg/ml was prepared in OPK buffer. Non-specific activity was evaluated using an isotype control antibody with a 3-fold dilution series from 30 to 3.33 μg/ml. To assess functionality of serum anti-Psl antibodies, heat-inactivated (56° C., 30 min) serum samples were prepared in a 10-fold dilution series from 1:100 to 1:1,000,000. OPK was determined in all assays at 2 h after P. aeruginosa addition.

### Cytotoxicity Assay

Cytotoxicity of P. aeruginosa 6077 strain and NCFBE clinical isolates in A549 epithelial cells and effects of MEDI3902 or endogenous anti-PcrV was investigated as described previously (DOI: 10.1093/infdis/jiv436), with some modifications. To determine endogenous anti-PcrV antibody functionality, a 3-fold dilution series of heat-inactivated serum from 1:100 to 1:24300 was utilized. Competition of endogenous anti-PcrV antibodies with MEDI3902 was assessed using serum at 1:100 with a 5-fold MEDI3902 dilution series from 300 to 0.096 μg/ml. The CytoTox-ONE™ kit was used to determine A549 cell death according to manufacturer's instructions.

### Detection of Psl Expression in NCFBE Clinical Isolates

Psl expression in NCFBE clinical isolates was measured by indirect enzyme-linked immunosorbent assays (ELISAs) as previously described (DiGiandomenico et al., Sci Transl Med., 2014, 12:262—DOI:10.1126/scitranslmed.3009655), using anti-Psl mAb Cam003 (DiGiandomenico et al., J Exp Med., 2012, 209:1273-1287—DOI: 10.1084/jem.20120033) or MEDI3902.

Detection of PcrV Expression from Clinical Isolates

PcrV expression was evaluated from whole-cell lysates by Western immunoblot analysis after in vitro induction of the Type III Secretion System of Pseudomonas aeruginosa (T3S) as described elsewhere (Warrener et al., Antimicrobial Agents and Chemotherapy, 2014, 58:8—DOI: 10.1128/AAC.02643-14; DOI: 10.1093/infdis/jiv436).

### Example 1: Target Conservation of Psl and PcrV in BE-Derived Pseudomonas aeruginosa Isolates

### Psl Operon Genes in NCFBE Isolates

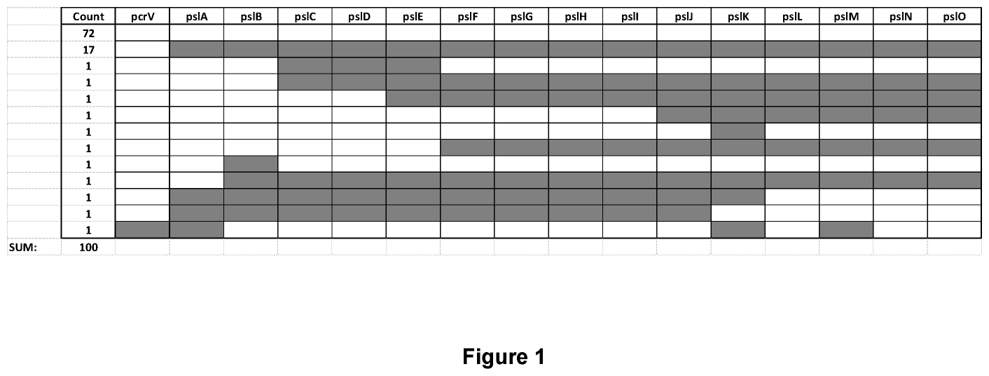

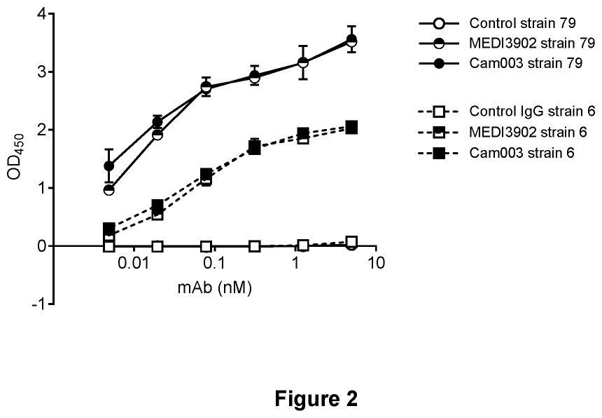

In sequenced isolates, 72 strains contained the full complement of psl operon genes (pslA through pslO). In contrast, 17 strains were missing the complete operon while 11 strains were missing one or more genes (FIG. 1). Representative isolates were tested in a whole-cell Psl ELISA with anti-Psl mAb Cam003 to identify which psl operon genes are required for Psl synthesis. Isolates that were either completely operon null, deficient for pslA, or lacking in genes for pslF-pslO were negative for Psl expression (FIG. 1). It had previously been shown that pslM-O were not required for Psl expression (DOI: 10.1093/infdis/jiy438). Strain 79 was null for pslK, but was shown to be bound by Cam003 and MEDI3902 but not a control IgG (FIG. 2). Strain 6 is an isolate containing a fully intact pslloci and served as a positive control for the assay (FIG. 2). In total, 73 of 100 isolates contained the genetic elements necessary to express Psl (FIG. 1).

The PcrV Gene is Highly Prevalent in NCFBE P. aeruginosa Isolates

A full-length pcrV gene was present in 99 of 100 of sequenced isolates demonstrating high prevalence among NCFBE-derived P. aeruginosa strains (FIG. 1). In a global surveillance study of acute infection-derived P. aeruginosa isolates, 46 PcrV variant sequences had previously been isolated using PAO1 PcrV as an amino acid sequence reference strain (DOI: 10.1093/infdis/jiy438). PcrV amino acid sequence analyses of NCFBE clinical isolates revealed substitutions at 18 positions, corresponding to 20 distinct PcrV full-length protein subtypes. While the majority of the PcrV variants from this strain collection were previously reported (DOI: 10.1093/infdis/jiy438), 6 new variant sequences (shown in bold in Table 2) were identified in NCFBE-derived P. aeruginosa isolates (Table 2).

TABLE 2

| | | Cluster |
|---|---|---|
| | Cluster ID | Count |
| 1 | Identical to NC_002516 (PAO1 PcrV) | 50 |
| 2 | L6F:S21P:S225K | 10 |
| 3 | L6F:A9G:S21P:S225R | 7 |
| 4 | S225R | 6 |
| 5 | L6F:A9G:S21P:S225K | 4 |
| 6 | A9G:S21P | 3 |
| 7 | L6F:S21P:S225R | 3 |
| 8 | S21P:K118E:D119A:S146G:V172L:D188G:D199G:D209N:S225R | 3 |
| 9 | S21P | 2 |
| 10 | V99A | 1 |
| 11 | S285I | 1 |
| 12 | S225G | 1 |
| 13 | E76D | 1 |
| 14 | L6F:A9G:S21P | 1 |
| 15 | A9G:S21P:S225R:V237I | 1 |
| 16 | L6F:A9S:S21P:S225R | 1 |
| 17 | L6F:A9G:S21P:D133E:S225R | 1 |
| 18 | S21P:K118E:D119A:S146G:V172L:D188G:D209N:S225R | 1 |
| 19 | L6F:A9G:S21P:L33M:S225R | 1 |
| 20 | L6F:A9G:S21P:E30K:S225R | 1 |
| | SUM: | 99 |

### Example 2: NCFBE PcrV Subtypes are Bound by Anti-PcrV MEDI3902 Binding Domain

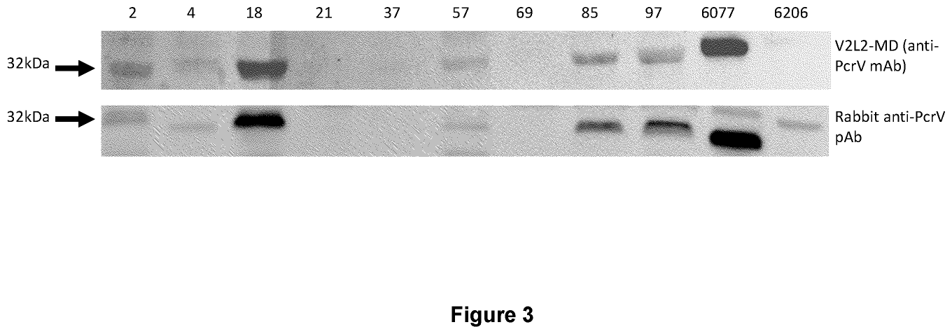

Although none of the newly identified amino acid substitutions were with the MEDI3902 anti-PcrV touch points, the ability of anti-PcrV mAb V2L2MD (which contains the complementarity determining regions, VH, and VL contained within MEDI3902) to bind PcrV expressed from whole cell lysates of subtype-specific strains cultured under type 3 secretion-inducing conditions was evaluated. Reactivity of lysates to purified polyclonal IgG derived from rabbits immunized with recombinant PcrV was performed to assess whether potential differences in protein reactivity were due to anti-PcrV mAb binding or to in vitro PcrV expression levels. Immunoblot analyses indicated that anti-PcrV mAb binding was comparable to the anti-PcrV polyclonal IgG binding against all newly identified PcrV variants that could express PcrV under in vitro conditions (FIG. 3). Overall, the data indicate that V2L2-MD bound to all variant PcrV sequences that were expressed in vitro.

### Example 3: MEDI3902 Maintains Functional Activity Against NCFBE-Derived P. aeruginosa Isolates

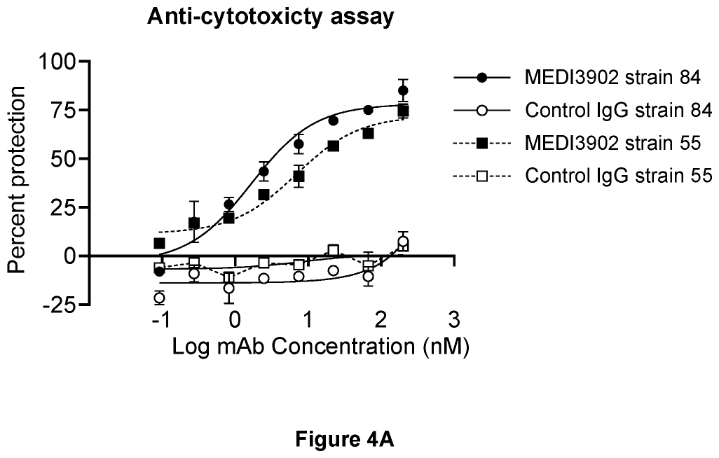

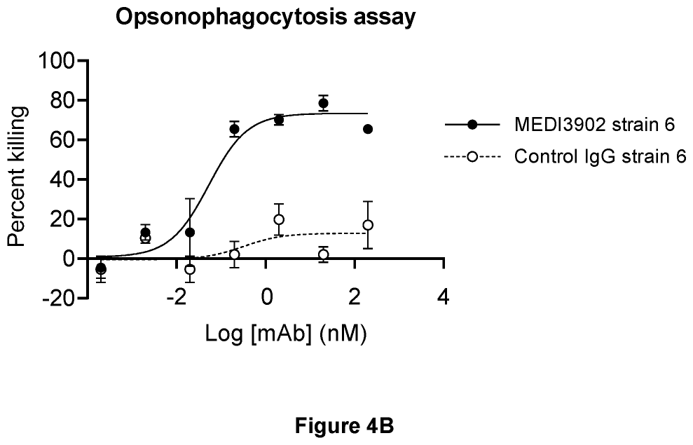

ExoU is a protein transported by the type III secretion system of Pseudomonas aeruginosa and is an important cytotoxin for this pathogen. MEDI3902 was evaluated for anti-cytotoxic activity against representative ExoU-producing P. aeruginosa NCFBE isolates (NCFBE strains 84 and 55), which exhibited cytotoxicity against an epithelial cell line (A549). MEDI3902 inhibited cellular intoxication of both strains, whereas a control IgG had no protective activity (FIG. 4A). In addition, an in vitro OPK assay was performed with a representative P. aeruginosa isolate that harbored a fully intact psl loci (NCFBE strain 6) and was shown to be bound by anti-Psl mAb Cam003 or MEDI3902. MEDI3902 and Cam003, but not control IgG, exhibited OPK activity against NCFBE isolate 6 (FIG. 4B).

### Example 4: MEDI3902 Enhances Killing of P. aeruginosa by Bronchiectasis Patient Peripheral Blood Neutrophils

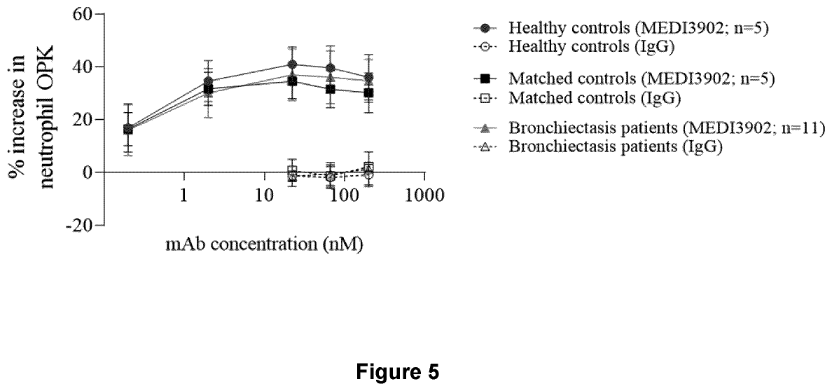

Neutrophil-mediated bacterial killing has previously been reported to be impaired in bronchiectasis. The ability of neutrophils to kill P. aeruginosa in the presence of increasing concentrations of MEDI3902 or an IgG control antibody was investigated, and compared with that in neutrophils from healthy controls and age- and sex-matched controls. Killing of P. aeruginosa was similar in all three participant groups (FIG. 5). 13ED3902 at the highest concentration utilized (200 nM) yielded an average increase in neutrophil-mediated P. aeruginosa killing of 34.6±8.1% (mean±SD); in healthy control neutrophils this was 36±8.6%, and in matched controls 30.1±7.6%. At an equimolar concentration (200 nM), addition of the control IgG antibody yielded no changes in killing in any of the groups (1.1±6.6%, −0.9±3.6%, 2.1±1.8%, respectively). Enhanced bacterial killing with MEDI3902 was observed down to the lowest concentration utilized (0.2 nM).

### Example 5: Endogenous Anti-Psl Antibodies were not Present in the Majority of Bronchiectasis Serum Samples, Whilst Most Patients Displayed Detectable Anti-PcrV

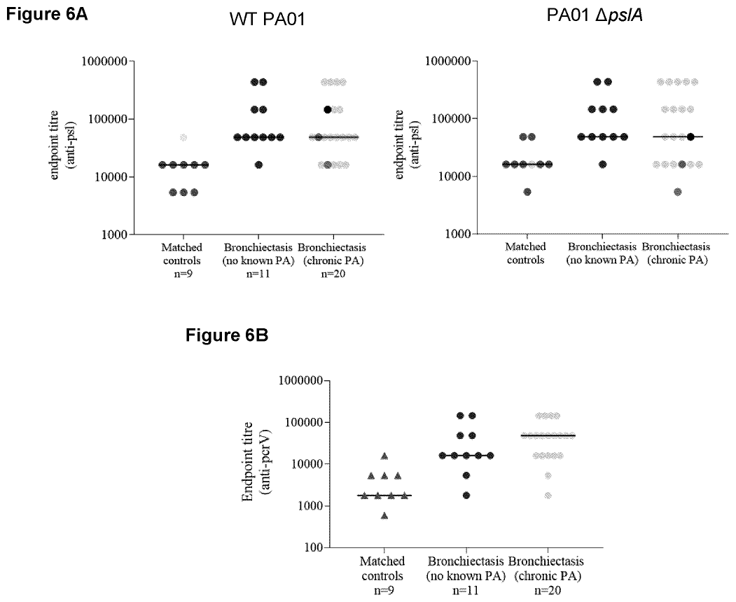

Previously, it has been reported that endogenous anti-Psl and PcrV serum IgG levels were variable in patients with acute P. aeruginosa infection and did not differ from those in non-Pseudomonas infection samples. In the present study, titres were determined in serum from bronchiectasis patients either with chronic P. aeruginosa airway infection or with no recent recorded infection, and from age- and sex-matched controls. Three out of thirty-one (31) bronchiectasis patients had detectable anti-Psl titres; all of these participants were part of the chronic infection group, whilst one (1) participant from the matched control group also had measurable anti-Psl (FIG. 6A). Overall, the bronchiectasis patient group appeared to demonstrate higher serum anti-Pseudomonas antibody levels than the matched control group. Anti-PcrV titres were detectable at varying levels in all bronchiectasis samples, with the chronic infection group demonstrating the highest titres (FIG. 6B).

### Example 6: Endogenous Anti-Psl Antibody Functional Activity was Variable in Bronchiectasis Patient Serum but Did not Compete with MEDI3902

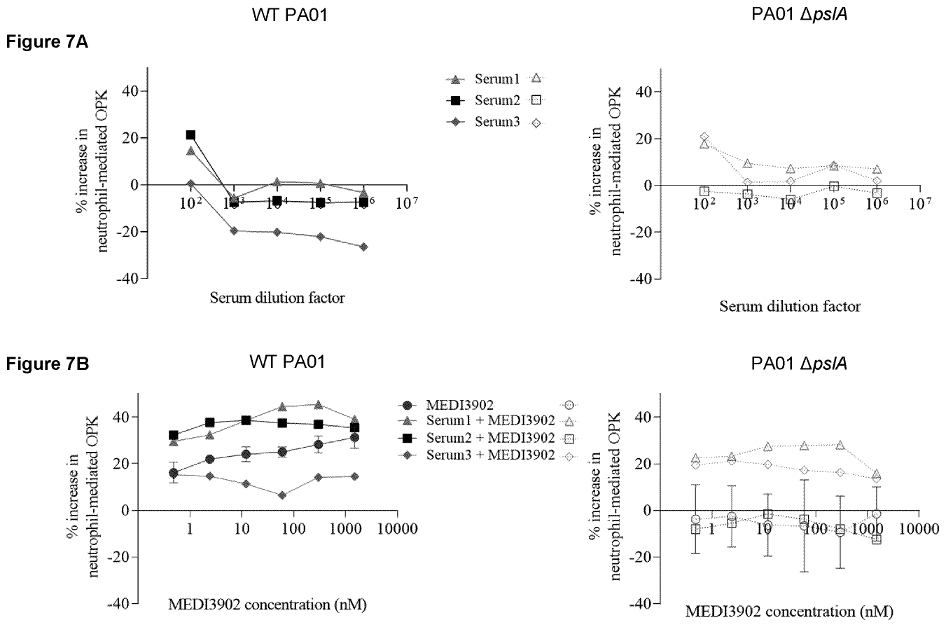

Three bronchiectasis patients demonstrated detectable serum anti-Psl antibody titres by ELISA. Functionality of endogenous anti-Psl antibody was tested in these samples for capacity to increase neutrophil-mediated killing of P. aeruginosa. Incubation of human neutrophils and wild-type (WT) P. aeruginosa with patient serum at a 1:100 dilution resulted in increased killing for 2 out of 3 samples (14.7% and 21.3% increases with serums 1 and 2, respectively; FIG. 7A (left panel)). Further serum dilutions did not demonstrate functional effects. When Psl-deficient (ΔpslA) P. aeruginosa was utilized in the same assay to determine the specific contribution of anti-Psl to these observed effects, the increased killing activity with serum 2 disappeared, whilst serum 1 retained its activity (FIG. 7A (right panel)). Only 1 out of 3 samples (serum 2) displayed functional anti-Psl antibodies, whilst effects on neutrophil-mediated killing in the other samples may be due to other factors.

The ability of endogenous anti-Psl to compete for ligand binding sites and potential to inhibit the effects of MEDI3902 was subsequently investigated by spiking patient serum at 1:100 with MEDI3902 at the indicated concentrations. MEDI3902 alone at all concentrations used, and MEDI3902 with each serum sample, demonstrated increased killing of WT P. aeruginosa (FIG. 7B (left panel)). These effects were abrogated in Psl-deficient bacteria (FIG. 7B (right panel)) when MEDI3902 alone or together with serum 2 was utilized. In serum 1 and 3, which did not exhibit functional anti-Psl, the increase in killing was maintained. This data indicates that endogenous anti-Psl antibodies in bronchiectasis patient serum, including potentially functional anti-Psl antibodies, does not appear to interfere or compete with MEDI3902.

### Example 7: Bronchiectasis Patient Serum with Endogenous Anti-PcrV had Protective Effects Against P. aeruginosa Cytotoxicity and Did not Impair MEDI3902 Activity

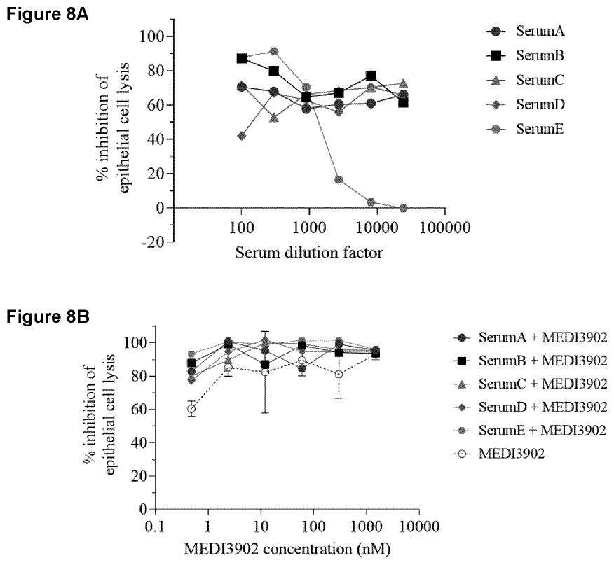

Since a high proportion of bronchiectasis patient serum samples contained detectable levels of anti-PcrV antibody, potential functionality of endogenous anti-PcrV was tested, with ability to inhibit the effect of the cytotoxic ExoU+6077 P. aeruginosa strain on A549 epithelial cell lysis as an output. All 5 serum samples tested demonstrated protective effects, on average inhibiting LDH release and therefore cell death with 6077 P. aeruginosa by 71.9±18.6% when added at a 1:100 dilution (FIG. 8A). Protective effects were seen up to the highest serum dilution of 1:24300 (53.2±30.1% inhibition of lysis) for all but 1 of these samples.

To determine whether endogenous anti-PcrV competes with MEDI3902 for binding and therefore alters activity, the experiment was repeated with serum at 1:100 dilution spiked with MEDI3902 at the indicated concentrations, or with MEDI3902 alone (FIG. 8B). No reduction in MEDI3902 activity at any of the concentrations used was observed in the presence of patient serum, and for most serum samples, anti-cytotoxic activity appeared to be additive when combined. In particular, for the lowest MEDI3902 concentration (0.48 nM), MEDI3902 alone yielded 60.4±4.5% protection, whilst MEDI3902 plus serum on average demonstrated 84.2±6.4% inhibition of cell lysis. This data indicates that endogenous anti-PcrV antibodies in bronchiectasis patient serum, including potentially functional anti-PcrV titers, do not interfere or compete with MEDI3902. In addition, this data indicates that the additional of MEDI3902 to serum has an additive anti-cytotoxic effect.

### Example 8: MEDI3902 in Patients with Bronchiectasis and Chronic Pseudomonas aeruginosa

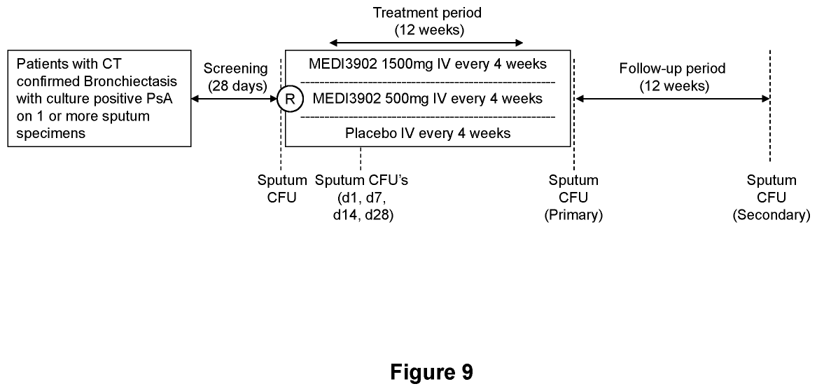

A randomized, double-blind phase 2 trial of MEDI3902 compared to placebo is conducted in participants with bronchiectasis and chronic Pseudomonas aeruginosa infection. A flow chart of the trial is shown in FIG. 9.

### Patients

Ninety (90) participants meeting the following inclusion criteria are randomized:
Age 18-85;
Clinical diagnosis of Bronchiectasis;
Previous CT scan of the chest demonstrating bronchiectasis in 1 or more lobes;
P. aeruginosa in sputum, bronchoalveolar lavage or another airway sample at least once in the 24 months prior to screening*
A sputum sample that is culture positive for P. aeruginosa sent at the screening visit and within 35 days of randomization*
a participant who does not have a previous positive sample for P. aeruginosa may submit two samples, at least 21 days apart, during the 35-day screening period. If these samples are both positive for P. aeruginosa then the last two inclusion criteria will be deemed met, and the patient may be enrolled

The participants are randomly allocated to receive either MEDI3902 1500 mg (IV, 4 weekly for 12 weeks), MEDI3902 500 mg (IV, 4 weekly for 12 weeks) or placebo (IV, 4 weekly for 12 weeks), all in addition to standard of care. Randomisation is 2:1:1, MEDI3902 1500 mg:MEDI3902 500 mg:placebo.

### Administration

MEDI3902 (1500 mg or 500 mg) or placebo (liquid buffer in saline) is administered by intravenous infusion (total volume of 250 mL) 4 weekly for 12 weeks.

### Assessment

Participants are asked to bring a spontaneous early morning sputum sample with them to visits. Sputum colour is assessed using the Murray Sputum Colour Chart. P. aeruginosa bacterial burden in sputum is assessed on days 7, 14, 28, 56, and 84.

Post bronchodilator spirometry at visits is carried out as using American Thoracic Society/European Respiratory Society's guidelines.

Forced expiratory volume in 1 second (FEV1) forced vital capacity (FVC) and Forced Expiratory Flow at 25-75% (FEF25-75) are measured.

Severity of bronchiectasis is evaluated using the Bronchiectasis Severity Index and MRC dyspnoea score. Exacerbation assessment uses the EMBARC definition of exacerbation. (Hill A T, et al., Eur Respir J. 2017; 49(6). doi:10.1183/13993003.00051-2017.)

Quality of life is assessed using the Quality of life bronchiectasis questionnaire (QOL-B) (Quittner A L et al. Quality of Life Questionnaire-Bronchiectasis: final psychometric analyses and determination of minimal important difference scores. Thorax. 2015; 70(1):12-20. doi:10.1136/thoraxjnl-2014-205918), the St. George's Respiratory Questionnaire (SGRQ) (Wilson C B, et al., Am J Respir Crit Care Med. 1997; 156(2 I):536-541. doi:10.1097/00008483-199803000-00011), and the Bronchiectasis Impact Measure (BIM) questionnaire (Crichton M L, et al., Eur Respir J. 2021; 57(5). doi:10.1183/13993003.03156-2020). The SGRQ has a 3-month recall period and is therefore performed at baseline and 3 months. The QOL-B and BIM questionnaire have a shorter recall period and are performed monthly.

### Results

An evaluation of P. aeruginosa bacterial burden in sputum at week 12 (day 84) demonstrates the efficacy of MEDI3902 in treating bronchiectasis in patients with chronic Pseudomonas aeruginosa infection.

Eradication of P. aeruginosa, defined by negative sputum cultures for P. aeruginosa at the end of treatment is also evaluated.

Changes from baseline in QOL-B and BIM questionnaires (days 28, 56, 84, and 168) and SGRQ (days 84 and 168) are evaluated to demonstrate the efficacy of MEDI3902 in improving the quality of life in bronchiectasis patients.

Occurrences of exacerbations (as per EMBARC definition of exacerbation; visit 1 to day 84) are evaluated to demonstrate the effect of MEDI3902 on time to first exacerbation.

Changes from baseline in FEV1 (day 28, 56, and 84) are evaluated to demonstrate the effect of MEDI3902 on pulmonary function.

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The breadth and scope of the present invention should not be limited by any of the above-described exemplary aspects, but should be defined only in accordance with the following claims and their equivalents.

1. A method of treating bronchiectasis in a subject in need thereof, the method comprising administering to the subject a bispecific antibody that specifically binds to Pseudomonas aeruginosa PcrV protein and Psl exopolysaccharide.

2. The method of claim 1, wherein administering the bispecific antibody that specifically binds to Pseudomonas aeruginosa PcrV protein and Psl exopolysaccharide improves pre-bronchodilator forced expiratory volume 1 (FEV_{I}) in the subject with bronchiectasis.

3. (canceled)

4. A The method of claim 1, wherein administering the bispecific antibody that specifically binds to Pseudomonas aeruginosa PcrV protein and Psl exopolysaccharide reduces bronchiectasis exacerbations in the subject with bronchiectasis.

5.-8. (canceled)

9. The method of claim 1, wherein the bronchiectasis is non-cystic fibrosis bronchiectasis.

10. (canceled)

11. The method of claim 1, wherein the subject is colonized with Pseudomonas aeruginosa, optionally wherein the respiratory tract of the subject is colonized with Pseudomonas aeruginosa.

12. The method of claim 11, wherein the Pseudomonas aeruginosa colonization has been detected by sputum culture.

13. The method of claim 1, wherein the subject is chronically infected with Pseudomonas aeruginosa.

14. The method of claim 1, wherein the subject has airway neutrophilia and/or sputum neutrophilia.

15. The method of claim 1, wherein the subject has sputum neutrophilia.

16.-24. (canceled)

25. The method of claim 1, wherein the administration eradicates Pseudomonas aeruginosa in the subject.

26.-27. (canceled)

28. The method of claim 1, wherein the bispecific antibody comprises a PcrV-binding domain comprising a heavy chain CDR1 comprising the amino acid sequence of SEQ ID NO: 1, a heavy chain CDR2 comprising the amino acid sequence of SEQ ID NO:2, a heavy chain CDR3 comprising the amino acid sequence of SEQ ID NO:3, a light chain CDR1 comprising the amino acid sequence of SEQ ID NO:4, a light chain CDR2 comprising the amino acid sequence of SEQ ID NO:5, and a light chain CDR3 comprising the amino acid sequence of SEQ ID NO:6.

29. The method of claim 28, wherein the bispecific antibody comprises a PcrV-binding domain comprising a heavy chain variable region comprising the amino acid sequence of SEQ ID NO: 13 and/or a light chain variable region comprising the amino acid sequence of SEQ ID NO: 14.

30.-32. (canceled)

33. The method of claim 1, wherein the bispecific antibody comprises a Psl-binding domain comprising a heavy chain CDR1 comprising the amino acid sequence of SEQ ID NO:7, a heavy chain CDR2 comprising the amino acid sequence of SEQ ID NO:8, a heavy chain CDR3 comprising the amino acid sequence of SEQ ID NO:9, a light chain CDR1 comprising the amino acid sequence of SEQ ID NO: 10, a light chain CDR2 comprising the amino acid sequence of SEQ ID NO: 11, and a light chain CDR3 comprising the amino acid sequence of SEQ ID NO: 12.

34. The method of claim 1, wherein the bispecific antibody comprises a Psl-binding domain comprising a heavy chain variable region comprising the amino acid sequence of SEQ ID NO: 15 and/or a light chain variable region comprising the amino acid sequence of SEQ ID NO: 16.

35. The method of claim 1, wherein the bispecific antibody comprises a Psl-binding domain with a heavy chain variable region and a light chain variable region on the same polypeptide.

36. The method of claim 1, wherein the bispecific antibody comprising a Psl-binding domain that is an scFv.

37.-39. (canceled)

40. The method of claim 36, wherein the scFv comprises the amino acid sequence of SEQ ID NO: 17.

41. The method of claim 1, wherein the bispecific antibody is an IgG antibody, optionally wherein the IgG antibody is an IgG1 antibody.

42. The method of claim 1, wherein the bispecific antibody comprises (i) a heavy chain of the formula VH-CH1-H1-L1-S-L2-H2-CH2-CH3, wherein VH is an anti-P. aeruginosa PcrV heavy chain variable domain; CH1 is a heavy chain constant region domain 1; H1 is a first heavy chain hinge region fragment; L1 is a first linker; S is an anti-P. aeruginosa Psl scFv molecule; L2 is a second linker; H2 is a second heavy chain hinge region fragment; CH2 is a heavy chain constant region domain-2; and CH3 is a heavy chain constant region domain-3; and (ii) a light chain of the formula VL-CL, wherein VL is an anti-P. aeruginosa PcrV light chain variable domain, and CL is an antibody light chain kappa constant region or an antibody light chain lambda region.

43. The method of claim 42, wherein the VH comprises the amino acid sequence of SEQ ID NO: 13, the scFv comprises the amino acid sequence of SEQ ID NO:15 and the amino acid sequence of SEQ ID NO: 16, and/or the VL comprises the amino acid sequence of SEQ ID NO:14.

44. The method of claim 42, wherein the VH comprises the amino acid sequence of SEQ ID NO: 13, the scFv comprises the amino acid sequence of SEQ ID NO:17, and/or the VL comprises the amino acid sequence of SEQ ID NO: 14.

45.-51. (canceled)

52. The method of claim 1, wherein the bispecific antibody is administered intravenously.
